(19) United States
(12) Patent Application Publication
(10) Pub. No.: US 2026/0348900 A1
(43) Pub. Date: Nov. 5, 2026

(54) BIOSENSOR WITH CONCAVE SENSING SURFACE

(71) Applicant: International Business Machines Corporation, Armonk, NY (US)

(72) Inventors: Viswas Sharad Purohit, Clifton Park, NY (US); Sufi Zafar, Briarcliff Manor, NY (US); Takashi Ando, Eastchester, NY (US); Christopher Peter D'Emic, Ossining, NY (US)

(21) Appl. No.: 19/194,082

(22) Filed: Apr. 30, 2025

(51) Int. Cl. G01N 27/327; G01N 27/30; G01N 33/50

(52) U.S. Cl. CPC G01N 27/3273; G01N 27/301; G01N 33/50

(57) ABSTRACT

Embodiments relate to a biosensor with concave sensing surface. As an aspect, the biosensor includes a transducer and a sensing structure coupled to the transducer and configured to receive an analyte. The sensing structure includes concave portions adjacent to raised portions, where a width of the concave portions ranges from 10-200 nanometers (nm), where a spacing between the concave portions ranges from 10-200 nm, and where a height of the raised portions ranges from 5-50 nm.

### BACKGROUND

The present invention generally relates to fabrication methods and resulting structures for integrated circuits (ICs), and more specifically, to fabrication methods and resulting structures configured and arranged to provide a biosensor with a concave sensing surface.

A biosensor is an analytical device, used for the detection of a chemical substance, that combines a biological component with a physicochemical detector. The sensitive biological element, e.g., tissue, microorganisms, organelles, cell receptors, enzymes, antibodies, nucleic acids, etc., is a biologically derived material or biomimetic component that interacts with, binds with, or recognizes the analyte under study. The biologically sensitive elements can also be created by biological engineering. The transducer or the detector element, which transforms one signal into another one, works in a physicochemical way in order the measure and quantify the results from the interaction of the analyte with the biological element. The biosensor reader device connects with the associated electronics or signal processors that are primarily responsible for the display of the results. A biosensor typically consists of a bio-receptor (e.g., enzyme, antibody, cell, nucleic acid, aptamer, etc.), transducer component (e.g., semiconducting material, nanomaterial, etc.), and electronic system which includes a signal amplifier, processor, and display. Transducers and electronics can be combined, for example, in CMOS-based microsensor systems. The recognition component, often called a bioreceptor, uses biomolecules from organisms or receptors modeled after biological systems to interact with the analyte of interest. This interaction is measured by the transducer which outputs a measurable signal proportional to the presence of the target analyte in the sample.

### SUMMARY

Embodiments of the present invention are directed to providing a biosensor with concave sensing surface. A biosensor includes a transducer and a sensing structure coupled to the transducer and configured to receive an analyte. The sensing structure comprises concave portions adjacent to raised portions. A width of the concave portions ranges from 10-200 nanometers (nm), and a spacing between the concave portions ranges from 10-200 nm. A height of the raised portions ranges from 5-50 nm.

According to one or more embodiments, a biosensor includes a transducer and a sensing structure coupled to the transducer and configured to receive an analyte. The sensing structure comprises concave portions adjacent to raised portions. A width of the concave portions ranges from 10-200 nanometers (nm), and a spacing between the concave portions ranges from 10-100 nm. A height of the raised portions ranges from 2-5 nm.

Other embodiments of the present invention implement features of the above-described devices/structures in methods and/or implement features of the methods in devices/structures.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a biosensor according to one or more embodiments;

FIGS. 2A and 2B depict cross-sectional and top views of a sensor structure according to one or more embodiments;

FIGS. 3A and 3B depict cross-sectional and top views of a sensor structure according to one or more embodiments;

FIGS. 4A and 4B depict cross-sectional and top views of a sensor structure according to one or more embodiments;

FIGS. 5A and 5B depict cross-sectional and top views of a sensor structure according to one or more embodiments;

FIGS. 6A and 6B depict cross-sectional and top views of a sensor structure according to one or more embodiments;

FIGS. 7A and 7B depict cross-sectional and top views of a sensor structure according to one or more embodiments;

FIG. 8 depicts a flow diagram of a method of forming a sensing structure with cavities having a sublithographic pitch according to one or more embodiments;

FIG. 9 depicts a flowchart of a method of configuring a biosensor according to one or more embodiments; and

FIG. 10 depicts a flowchart of a method of configuring a biosensor according to one or more embodiments.

### DETAILED DESCRIPTION

For the sake of brevity, conventional techniques related to semiconductor device and integrated circuit (IC) fabrication may or may not be described in detail herein. Moreover, the various tasks and process steps described herein can be incorporated into a more comprehensive procedure or process having additional steps or functionality not described in detail herein. In particular, various steps in the manufacture of semiconductor devices and semiconductor-based ICs are well known and so, in the interest of brevity, many conventional steps will only be mentioned briefly herein or will be omitted entirely without providing the well-known process details.

The MOSFET is a transistor used for amplifying or switching electronic signals. The MOSFET has a source, a drain, and a metal gate electrode. The metal gate is electrically insulated from the main semiconductor n-channel or p-channel by a thin layer of insulating material, for example, silicon dioxide or glass, which makes the input resistance of the MOSFET relatively high. The gate voltage controls whether the current path from the source to the drain is an open circuit (“off”) or a resistive path (“on”). N-type field effect transistors (NFET) and p-type field effect transistors (PFET) are two types of complementary MOSFETs. The NFET includes n-doped source and drain junctions and uses electrons as the current carriers. The PFET includes p-doped source and drain junctions and uses holes as the current carriers.

The nanowire or nanosheet MOSFET is a type of MOSFET that uses multiple stacked nanowires/nanosheets to form multiple channel regions. The gate regions of a nanosheet MOSFET are formed by wrapping gate stack materials around the multiple nanowire/nanosheet channels. This configuration is known as a gate-all-around (GAA) FET structure. The nanowire/nanosheet MOSFET device mitigates the effects of short channels and reduces the drain-induced barrier.

Biosensors are electronic sensors that include transistors as the transducer used to detect the electrical current, voltage, capacitance, etc., associated with the test solution or analyte. The transistor as the transducer can be a field-effect transistor (FET), a bipolar junction transistor (BJT), a metal-oxide semiconductor field-effect transistor (MOSFET), etc. The sensitivity of an electronic sensor depends on the transducer design, the sensing surface, and the reference electrode.

The present disclosure provides a novel sensing surface topography to achieve increased sensitivity for biosensors used to analyze a test solution or analyte. According to one or more embodiments, sensing structures are disclosed with concave surfaces having higher sensitivity than flat surfaces. One or more embodiments provide sensing surfaces with a concave topography and methods for fabrication.

On the sensing surface, the solid to liquid interfaces have double layers with thicknesses of Debye length (λ), where the Debye length λ value depends inversely on the ionic concentration of the test solution. The Debye length λ is a measure of the distance over which mobile charge carriers such as electrons or ions screen out electric fields in a conductive medium, such as an electrolyte solution. In biosensors, the Debye length λ is particularly useful because it affects the sensitivity of the sensor to detect charges, particularly in high ionic concentration environments where charge screening can limit detection capabilities. One or more embodiments provide sensing structures designed to increase the Debye length λ, thereby improving the functioning of the biosensor.

Charge screening effects (e.g., α exp ^{z/λ}) increases with increasing distance (z) from the solid surface. Potentiometric biosensors (e.g., FET biosensors) detect biomolecules by detecting their charges. Accordingly, charge screening reduces sensitivity for charges located at z>1. This can limit detection of large proteins in high ionic concentration solutions (e.g., blood with λ approximately 10 angstroms (A)). In accordance with one or more embodiments, a novel aspect of the present disclosure is that sensing surfaces be patterned with concave surface vertical vias and/or lateral trenches to increase the Debye length, thus overcoming the charge screening issues and increasing sensor sensitivity.

The present disclosure provides various topographies for the sensing structure that is coupled to the transducer. One or more embodiments include the sensing surface with two metals on cylindrical concave structures. The metals are selected such that biolayer selectively binds to the first metal of the via walls (e.g., vertical surfaces) but not to flat surfaces. Therefore, the sensing surface has a combination of concave and flat surfaces.

Also, one or more embodiments disclose the sensing surface with fin arrays having lateral channel layers with two different materials. The materials are selected such that biolayer selectively binds to the first material of fin sidewalls (e.g., vertical surfaces) but not to flat surface at the top of the fin sidewalls. The sensing surface can have two different metals on the fin structures in which the metals are selected such that biolayer selectively binds to first metal (e.g., fin sidewalls) but not to flat surfaces.

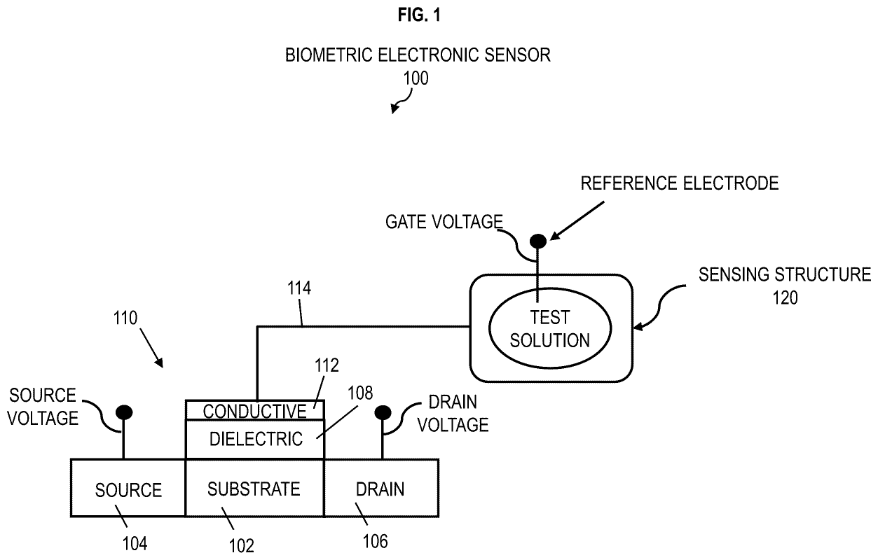

Turning now to a more detailed description of aspects of the present disclosure, FIG. 1 depicts a block diagram of an example illustration of a biosensor 100. The biosensor 100 includes a transducer 110 coupled to a sensing structure 120. The transducer 110 includes a transistor. The transistor includes a substrate 102, a source 104, a drain 106, a gate dielectric 108 such as a high-k dielectric material, and a conductive material 112 such as one or more gate work function metal/materials. A connector 114 can connect the sensing structure 120 to the transducer 110. In one or more embodiments, the sensing structure 120 can be positioned on and electrically connected to the transducer 110 with the connector 114 omitted. Although FIG. 1 illustrates an example transistor design for the transducer 110, any transistor design having its gate connected to and/or controlled by the electrical current, capacitance, voltage of the sensing structure 120 can be utilized for transducer 110.

Standard semiconductor fabrication techniques can be utilized to fabricate the transducer 110 and the sensing structure 120 as understood by one of ordinary skill in the art. Any suitable lithography processes including deposition techniques and etching techniques can be utilized herein.

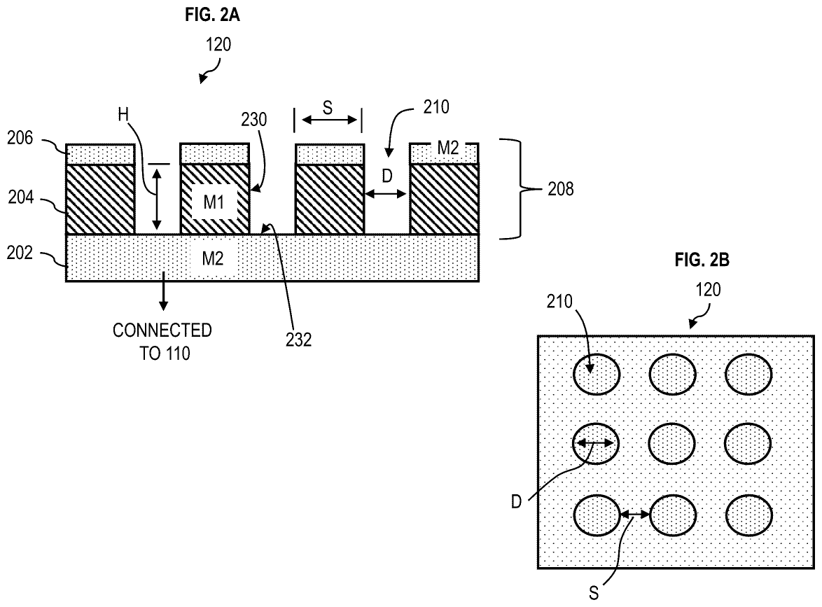

Examples of the concave topology of the sensing structure 120 are discussed further in FIGS. 2A and 2B, FIGS. 3A and 3B, FIGS. 4A and 4B, FIGS. 5A and 5B, FIGS. 6A and 6B, and 7A and 7B according to one of more embodiments.

FIGS. 2A and 2B depict a cross-sectional view and a top view of an example of the sensing structure 120 according to one or more embodiments. In this example, the sensing structure 120 includes two different materials (e.g., metals) on cylindrical concave structures. In FIGS. 2A and 2B, a first layer 202 of the sensing structure 120 is connected to the transducer 110. A second layer 204 is formed on the first layer 202 and a third layer 206 is formed on top of the second layer 204. The third layer 206 and the second layer 204 are patterned using lithography into a pattern to form pillars 208 (e.g., raised portions). The pillars 208 include the (patterned) second and third layers 204 and 206.

Cavities 210 (e.g., concave portions) formed by the pillars 208 of the (patterned) second and third layers 204 and 206 have a width or diameter D and a height H. The height H corresponds to the height of the second layer 204 that is the sensing surface. The spacing S between the cavities 210 corresponds to the width of the pillars 208. The width or diameter D of the cavities 210 can range from 10-200 nanometers (nm). The cavity height H or height of the pillars 208 can range from about 5-50 nm. The spacing S between the cavities 210 or the width of pillars 208 can range from about 10-100 nm. In accordance with embodiments, there are many technical benefits and solutions provided by the concave topology of the sensing structure 120 discussed herein. It is determined that charge screening effects decrease with increasing cavity width and start to become negligible for a width or diameter greater than (>) 200 nm. Hence, in accordance with one or more embodiments, the upper limit for width is 200 nm. The lower width limit is 10 nm because the majority of proteins are greater than (<)10nm. If the cavity width is smaller than 10 nm, then most proteins will not be able to enter the cavity for detection. Further, choosing a width or diameter D ≈10-200 nm, height H≈5-50 nm, and spacing S≈10-100 nm keeps every cavity smaller than, or on the order of, the Debye length in physiological buffers and the electric-double-layer fills the entire concavity; thus the target-induced charge shifts are not screened, yielding a greater than or equal to (≥)2×signal-to-noise boost versus larger or deeper concavities.

The second layer 204 is formed of a first material, and the first layer 202 is formed of a second material different from the second material. The first layer 202 and the second layer 204 can be formed of different metals. The materials of the first layer 202 and the third layer 206 can be the same. In one or more embodiments, the first layer 202 and the third layer 206 can be different materials. Whether the materials of the first layer 202 and the third layer 206 are the same or different, the materials of the first layer 202 and the third layer 206 are not the same as the material of the second layer 204. Example materials of the first layer 202 and third layer 206 can include titanium oxide (TiOₓ), titanium nitride (TiN), platinum (Pt), gold (Au), etc. The first layer 202 is connected to the transducer 110 to provide electrical current, capacitance, gate voltage, etc.

The second layer 204 acts as the probe of the sensing structure 120 such that test biomolecules of the test solution selectively attach to and selectively bind to the walls 230 of the second layer 204 forming pillars 208 but not the flat surfaces 232 of the first layer 202 or the flat surfaces of the third layer 206. Example materials of the second layer 204 can include ruthenium, ruthenium oxide, aluminum and its alloys (e.g., Al—Cu, Al—Si—Cu), tungsten, molybdenum, copper, platinum, iridium oxide (IrO₂), titanium nitride (TiN), zirconium oxide (ZrO₂), hafnium oxide (HfO₂), etc., such that ligands or ligand molecules can be selectively attached to the material of the second layer 204 but not materials of the first and third layers 202 and 206 in order for the test biomolecules of the test solution to bind to the ligands on the walls 230 of the second layer 204. In one example, a probe layer of phosphonic acid-ligand molecules can be selectively attached to the ruthenium oxide of the second layer 204 but not to materials of the first and third layers 202 and 206. Accordingly, the materials (which can be the same or different) of the first and third layers 202 and 206 do not contribute to the electrical current, capacitance, and/or voltage associated with the test biomolecules of the test solution.

By only having the sensing surface be the walls 230 of the pillars 208 of the second layer 204 and not the flat surfaces 232 of the first layer 202 as depicted in FIGS. 2A and 2B, the Debye length λ is increased, thereby improving the function of the biosensor 100 itself. In an example estimate of the sensitivity enhancement using the via walls 230 (only), where the width or diameter D is 30 nm, the spacing S is 30 nm, and the distance (z) from the solid sensing surface is approximately 9 nm, there is a sensitivity increase of approximately 11 times greater for the biosensor 100 as compared to having only flat surfaces as the sensing structure. The typical antibody size is about 10 nm, where the antibody is the test biomolecule.

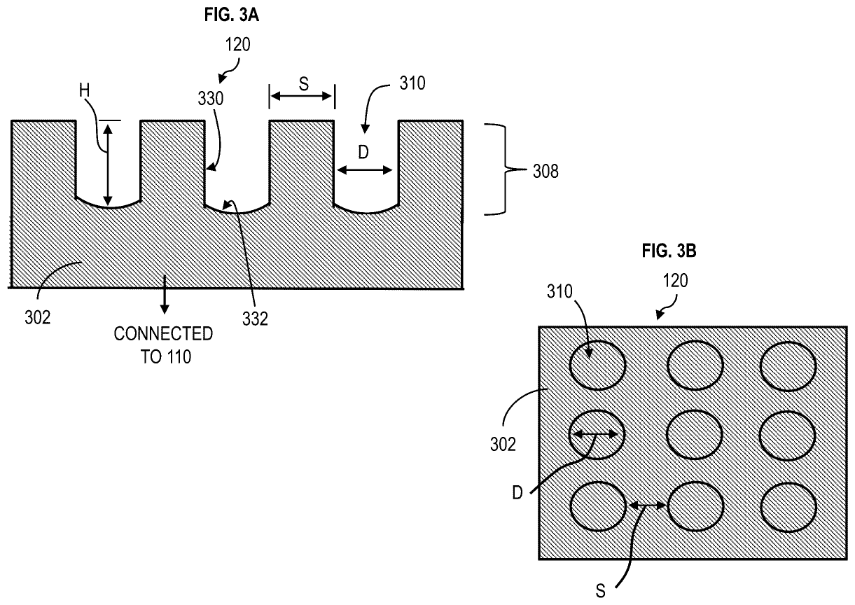

FIGS. 3A and 3B depict a cross-sectional view and a top view of an example of the sensing structure 120 according to one or more embodiments. In this example, the sensing structure 120 includes one metal on cylindrical concave structures. In FIGS. 3A and 3B, a layer 302 is connected to the transducer 110. The layer 302 is patterned using lithography into a pattern to form pillars 308 (e.g., raised portions). The pillars 308 include the pattern of the layer 302.

Cavities 310 (e.g., concave portions) are formed by patterning the pillars 308 in the layer 302 and have a width or diameter D and a height H. The spacing S between the cavities 310 corresponds to the width of the pillars 308. The width or diameter D of the cavities 310 can range from 10-200 nanometers (nm). The cavity height H or height of the pillars 308 can range from about 5-50 nm. The spacing S between the cavities 310 or the width of pillars 308 can range from about 10-200 nm.

The layer 302 is formed of a metal. Example materials of the layer 302 can include titanium oxide (TiOₓ), titanium nitride (TiN), platinum (Pt), gold (Au), etc. The layer 302 is connected to the transducer 110 to provide electrical current, capacitance, gate voltage, etc.

The sensing structure 120 includes walls 330 forming the cavities 310 and pillars 308 and includes flat surfaces 332 at the bottom of the cavities. For determining the sensitivity of the sensing structure 120, the sensing of sensing structure 120 is a combination of the walls 330 of the cavities 310 (which are concave portions) and flat surfaces 332. By having the sensing surface be both the (cavity/pillar) walls 330 of the pillars 308 and the flat surfaces 332 of the layer 302, the Debye length is increased over having a sensing surface with an entirely flat surface, thereby improving the function of the biosensor 100 itself. In an example estimate of the sensitivity enhancement using the (cavity/pillar) walls 330 in addition to the flat surfaces 332 as depicted in FIGS. 3A and 3B, where the width or diameter D is 30 nm, the spacing S is 30 nm, and the distance (z) from the solid sensing surface is approximately 9 nm, there is a sensitivity increase of approximately 4 of 5 times greater for the biosensor 100 compared to only having a flat surface. The typical antibody size is about 10 nm, where the antibody is the test biomolecule.

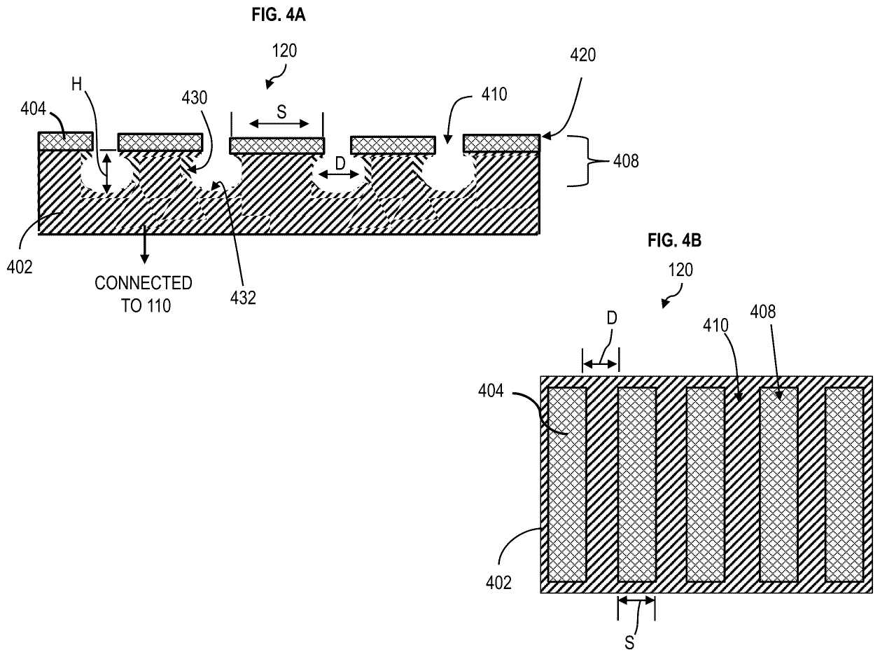

FIGS. 4A and 4B depict a cross-sectional view and a top view of an example of the sensing structure 120 according to one or more embodiments. In this example, the sensing structure 120 includes a fin array with a lateral channel layer with two different materials. In FIGS. 4A and 4B, a first layer 402 is connected to the transducer 110. A second layer 404 is formed on the first layer 402. The second layer 404 is patterned into a fin mask, and the pattern of the fin mask is utilized to etch the first layer 402. The first layer 402 and second layer 404 are patterned using lithography to form and array of fins 408 (e.g., raised portions). The fins 408 include a portion of the first layer 402 and the second layer 404.

Cavities 410 (e.g., concave portions) as lateral trenches are formed by patterning the fins 408 in the first and second layers 402 and 404, and cavities 410 have a width or diameter D and a height H. The height H corresponds to the height of the first layer 402 that is portion of the fins 408 and forms walls of the sensing surface of the fins 408. The spacing S between the cavities 410 (or lateral trenches) corresponds to the width of the fins 408. The width or diameter D of the cavities 410 can range from 10-200 nanometers (nm). The cavity height H or height of the fins 408 can range from about 2-5 nm. The spacing S between the cavities 410 or the width of fins 408 can range from about 10-100 nm. As further technical benefits and solutions, selecting an ultra-shallow trench height of 2-5 nm keeps every analyte molecule within a single Debye length of the electrode, eliminating charge-screening that plagues deeper cavities; for example, deep concavities (>10 nm deep) lose signal, whereas this geometry of embodiments delivers a stronger, low-noise response that enables sub-attomolar detection in physiological buffers.

The sensing structure 120 includes walls 430 (e.g., fin sidewalls) forming the cavities 410 (e.g., lateral trenches) and fins 408 and includes flat surfaces 432 at the bottom of the cavities. The first layer 402 acts as the probe of the sensing structure 120 such that test biomolecules of the test solution selectively attach to and selectively bind to the walls 430 and the flat surfaces 232 of the first layer 402 forming fins 408 but not to the second layer 404. The first layer 402 is formed of a metal. Example materials of the first layer 402 can include ruthenium (Ru), ruthenium oxide (RuOₓ), titanium (Ti), titanium oxide (TiOₓ), titanium nitride (TiN), platinum (Pt), gold (Au), etc. The first layer 402 is connected to the transducer 110 to provide electrical current, capacitance, gate voltage, etc. Material of the first layer 402 can be selected such that ligands or ligand molecules can be selectively attached to the material of the first layer 402 but not material of the second layer 404 in order for the test biomolecules of the test solution to bind to the ligands on the first layer 402.

The material of the second layer 404 is different from the first layer 402. The second layer 404 can be a different metal from the first layer or an insulator. Example materials for the second layer 404 may include silicon nitride (SiN), silicon dioxide (SiO₂), silicon carbide (SiC), polysilicon (doped or intrinsic), aluminum nitride (AlN), molybdenum nitride (MoN), porous SiO₂/SiCOH, etc. In one or more embodiments, the second layer 404 may serve as a softmask or hardmask when patterning the first layer 402.

For determining the sensitivity of the sensing structure 120, the sensitivity of sensing structure 120 is a combination of the walls 430 (e.g., fin sidewalls or raised portions) of the cavities 410 (which are concave portions) and flat surfaces 432. By having the sensing surface be both the walls 430 of the fins 408 and the flat surfaces 432 of the first layer 402, the Debye length λ is increased over having a sensing surface with only flat surfaces, thereby improving the function of the biosensor 100 itself.

In an example estimate of the sensitivity enhancement using the walls 430 (e.g., fin sidewalls) in addition to the flat surfaces 432 as depicted in FIGS. 4A and 4B, where the width or diameter D is 30 nm, the spacing S is 30 nm, and the distance (z) from the solid sensing surface is approximately 9 nm, there is a sensitivity increase of approximately 8 times greater for the biosensor 100 as compared to an entirely flat surface. The typical antibody size is about 10 nm, where the antibody is the test biomolecule.

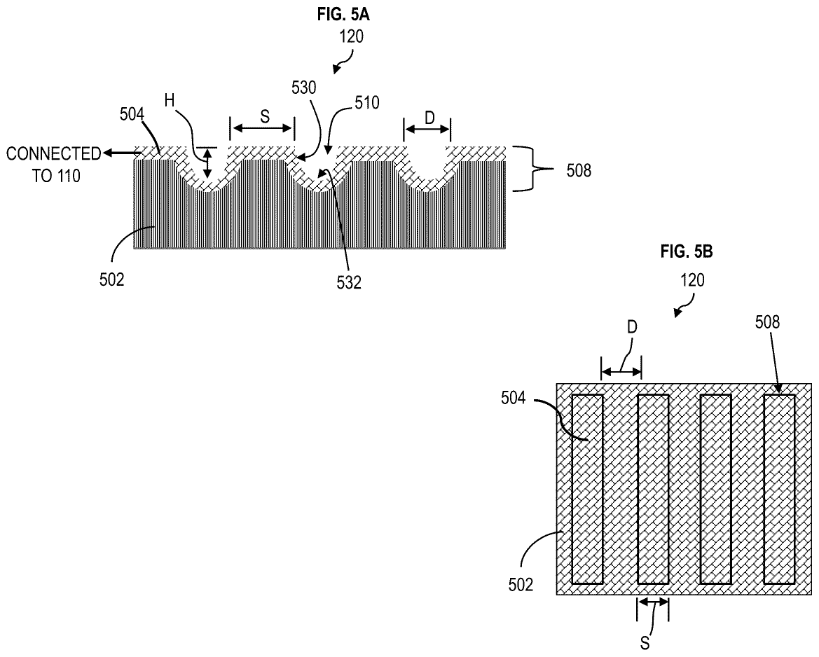

FIGS. 5A and 5B depict a cross-sectional view and a top view of an example of the sensing structure 120 according to one or more embodiments. In this example, the sensing structure 120 includes a fin array with lateral channels having the same material. In FIGS. 5A and 5B, a first layer 502 is connected to the transducer 110. A second layer 504 is conformally formed on the first layer 502. The sensing structure 120 has cavities 510 as lateral trenches and fins 508 as raised portions. The cavities 410 (e.g., concave portions) are formed by patterning the fins in the first layer 502, and the second layer 504 is conformally formed on the troughs and crests in the first layer 502, thereby forming the cavities 510 and fins 508. The fins 508 include the second layer 504 and a portion of the first layer 502. The cavities 510 have a width or diameter D and a height H. The height H of the cavities 510 is the height of the second layer 504 forming the fins 508. The spacing S between the cavities 510 (or lateral trenches) corresponds to the width of the fins 508. The width or diameter D of the cavities 510 can range from 10-200 nanometers (nm). The cavity height H or height of the fins 508 can range from about 2-5 nm. The spacing S between the cavities 510 or the width of fins 508 can range from about 10-100 nm. As noted above for technical benefits and solutions, setting the cavity height H to only 2-5 nm keeps every bound analyte within a single Debye length of the electrode, virtually eliminating charge-screening; deeper concavities (>10 nm) suffer significant signal loss; thus, this ultra-shallow profile delivers a markedly stronger, low-noise response, in accordance with one or more embodiments.

The sensing structure 120 includes walls 530 (e.g., fin sidewalls or raised portions) forming the cavities 510 (e.g., lateral trenches or concave portions) and fins 508 and includes flat surfaces 532 at the bottom of the cavities 510. The second layer 504 acts as the probe of the sensing structure 120 such that test biomolecules of the test solution selectively attach to and selectively bind to the walls 530 and the flat surfaces 532 of the second layer 504 forming fins 508. The first layer 502 is formed of a dielectric material that is non-conducting such as silicon nitride (SiN), silicon dioxide (SiO₂), silicon oxynitride (SiON), aluminum oxide (Al₂O₃), hafnium oxide (HfO₂), zirconium oxide (ZrO₂), etc. Example materials of the second layer 504 can include ruthenium (Ru), ruthenium oxide (RuOₓ), titanium (Ti), titanium oxide (TiOₓ), titanium nitride (TiN), platinum (Pt), gold (Au), etc. The second layer 504 is connected to the transducer 110 to provide electrical current, capacitance, gate voltage, etc. Material of the second layer 504 can be selected such that ligands or ligand molecules can be selectively attached to the material in order for the test biomolecules of the test solution to bind to the ligands on the first layer 402. The material of the second layer 504 is different from the first layer 502.

For determining the sensitivity of the sensing structure 120, the sensing of sensing structure 120 is a combination of the walls 530 (e.g., fin sidewalls) of the cavities 510 (which are concave portions) and flat surfaces 532. By having the sensing surface be both the walls 530 of the fins 508 and the flat surfaces 532 of the second layer 504, the Debye length λ is increased over having a sensing surface with only flat surfaces, thereby improving the function of the biosensor 100 itself.

In an example estimate of the sensitivity enhancement using the walls 530 (e.g., fin sidewalls) in addition to the flat surfaces 532 as depicted in FIGS. 5A and 5B, where the width or diameter D is 30 nm, the spacing S is 30 nm, and the distance (z) from the solid sensing surface is approximately 9 nm, there is a sensitivity increase of approximately 2 times greater for the biosensor 100 as compared to an entirely flat surface. The typical antibody size is about 10 nm, where the antibody is the test biomolecule.

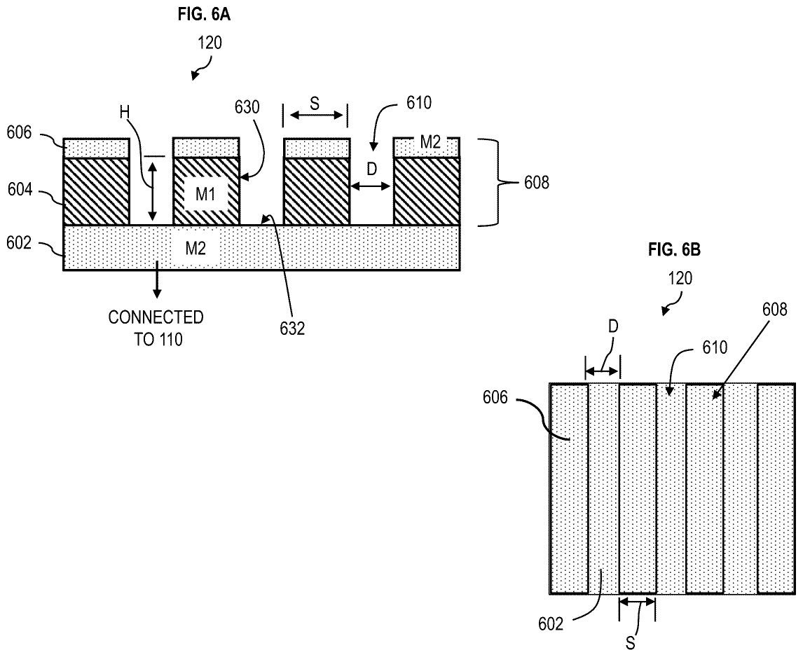

FIGS. 6A and 6B depict a cross-sectional view and a top view of an example of the sensing structure 120 according to one or more embodiments. In this example, the sensing structure 120 includes two different materials (e.g., metals) on lateral channels as concave structures. In FIGS. 6A and 6B, a first layer 602 of the sensing structure 120 is connected to the transducer 110. A second layer 604 is formed on the first layer 602 and a third layer 606 is formed on top of the second layer 604. The third layer 606 and the second layer 604 are patterned using lithography into a pattern to form fins 608 as the raised portions. The fins 608 include the (patterned) second and third layers 604 and 606.

Cavities 610 (e.g., concave portions) as lateral trenches are formed by the fins 608 of the (patterned) second and third layers 604 and 606 have a width or diameter D and a height H. The height H corresponds to the height of the second layer 604 that is the sensing surface. The spacing S between the cavities 610 corresponds to the width of the fins 608. The width or diameter D of the cavities 610 can range from about 10-200 nanometers (nm). The cavity height H or height of the fins 608 can range from about 2-5 nm. The spacing S between the cavities 610 or the width of fins 608 can range from about 10-100 nm.

The second layer 604 is formed of a first material, and the first layer 602 is formed of a second material different from the second material. The first layer 602 and the second layer 604 can be formed of different metals. The materials of the first layer 602 and the third layer 606 can be the same. In one or more embodiments, the first layer 602 and the third layer 606 can be different materials. Whether the materials of the first layer 602 and the third layer 606 are the same or different, the materials of the first layer 602 and the third layer 606 are not the same as the material of the second layer 604. Example materials of the first layer 602 and third layer 606 can include titanium oxide (TiOₓ), titanium nitride (TiN), platinum (Pt), gold (Au), etc. The first layer 602 is connected to the transducer 110 to provide electrical current, capacitance, gate voltage, etc.

The second layer 604 acts as the probe of the sensing structure 120 such that test biomolecules of the test solution selectively attach to and selectively bind to the walls 630 (e.g., fin sidewalls or raised portions) of the second layer 604 forming fins 608 but not the flat surfaces 632 of the first layer 602 or the third layer 606. Example materials of the second layer 604 can include ruthenium, ruthenium oxide, etc., such that ligands or ligand molecules can be selectively attached to the material of the second layer 604 but not materials of the first and third layers 602 and 606 in order for the test biomolecules of the test solution to bind to the ligands on the walls 630 of the second layer 604. In one example, a probe layer of phosphonic acid-ligand molecules can be selectively attached to the ruthenium oxide of the second layer 604 but not to materials of the first and third layers 602 and 606. Accordingly, the materials (which can be the same or different) of the first and third layers 602 and 606 do not contribute to the electrical current, capacitance, and/or voltage associated with the test biomolecules of the test solution.

By only having the sensing surface be the walls 630 of the fins 608 of the second layer 604 and not the flat surfaces 632 of the first layer 602 or third layer 606 as depicted in FIGS. 6A and 6B, the Debye length λ is increased, thereby improving the function of the biosensor 100 itself. In an example estimate of the sensitivity enhancement using the via walls 230 (only), where the width or diameter D is 30 nm, the spacing S is 30 nm, and the distance (z) from the solid sensing surface is approximately 9 nm, there is a sensitivity increase for the biosensor 100 as compared to having an entirely flat surface. The typical antibody size is about 10 nm, where the antibody is the test biomolecule.

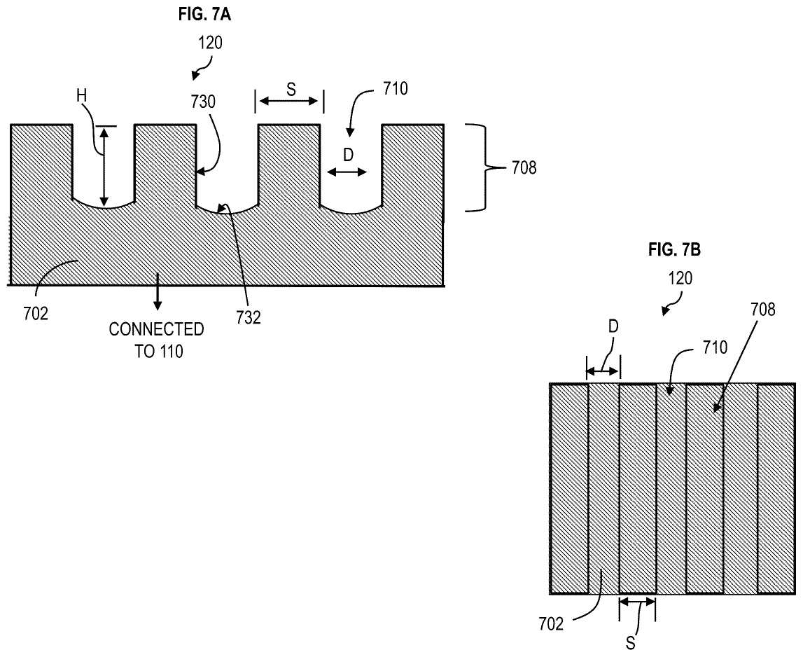

FIGS. 7A and 7B depict a cross-sectional view and a top view of an example of the sensing structure 120 according to one or more embodiments. In this example, the sensing structure 120 includes metal of fin structures. In FIGS. 7A and 7B, a layer 702 is connected to the transducer 110. The layer 702 is patterned using lithography into a pattern to form fins 708 (e.g., raised portions). The fins 708 include the pattern of the layer 702.

Cavities 710 (e.g., concave portions) as lateral trenches are formed by patterning the fins 708 in the layer 302 and have a width or diameter D and a height H. The spacing S between the cavities 710 corresponds to the width of the fins 708. The width or diameter D of the cavities 710 can range from about 10-100 nanometers (nm). The cavity height H or height of the fins 708 can range from about 2-5 nm. The spacing S between the cavities 710 or the width of fins 708 can range from about 10-100 nm.

The layer 702 is formed of a metal. Example materials of the layer 702 can include titanium oxide (TiOₓ), titanium nitride (TiN), platinum (Pt), gold (Au), etc. The layer 302 is connected to the transducer 110 to provide electrical current, capacitance, gate voltage, etc.

The sensing structure 120 includes walls 730 (e.g., fin sidewalls) forming the cavities 710 and fins 708 and includes flat surfaces 732 at the bottom of the cavities. For determining the sensitivity of the sensing structure 120, the sensing of sensing structure 120 is a combination of the walls 730 of the cavities 710 (which are concave portions) and flat surfaces 732. By having the sensing surface be both the (fin) walls 730 of the fins 708 and the flat surfaces 732, the Debye length λ is increased over having a sensing surface with an entirely flat surface, thereby improving the function of the biosensor 100 itself.

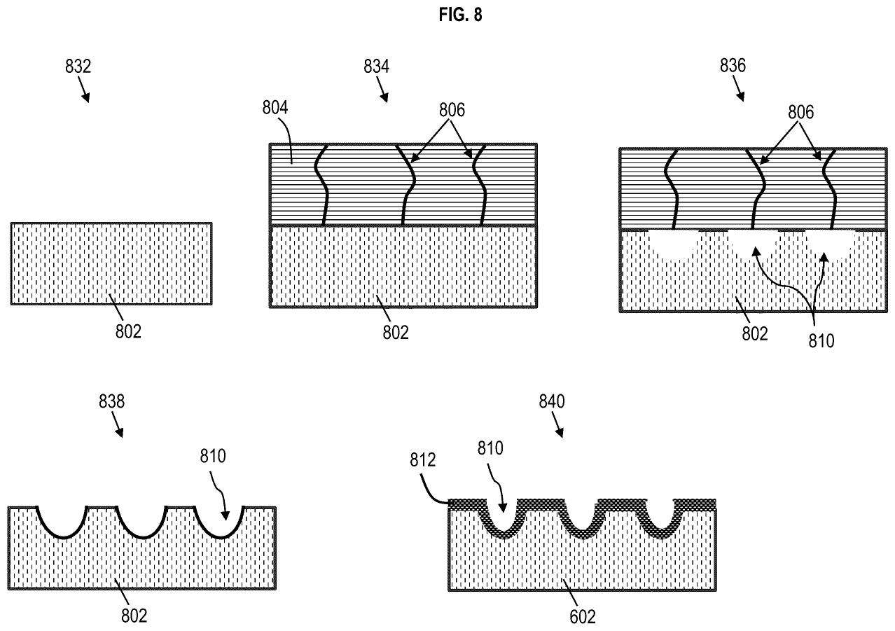

FIG. 8 depicts a flow diagram of a method to form a sensing structure with cavities having a sublithographic pitch according to one or more embodiments. FIG. 8 depicts a method of fabricating using columnar grains, particularly TiN column grains.

View 832 illustrates a first layer 802. The first layer 802 can be a dielectric material such as, for example, silicon dioxide (SiO2), etc.

In view 834, a second layer 804 is formed on top of the first layer with columnar grains 806 extending vertically from top to bottom. The second layer 804 can be titanium nitride. The size of the columnar grains 806 can be controlled by temperature. Columnar grains refer to a specific microstructural feature observed in crystalline materials, particularly in thin films and coatings. These columnar grains are elongated and have a column-like shape, extending vertically through the thickness of the material. Columnar grains are formed during certain deposition processes, such as physical vapor deposition (PVD) or chemical vapor deposition (CVD), where the growth conditions favor the development of elongated grain structures.

In view 836, isotropic etching (e.g., with diluted hydrofluoric (HF) acid) through the columnar grains 806 is performed. The isotropic etching forms cavities 810 or concave dents in the first layer 802.

In view 838, the second layer 804 is removed. For example, the titanium nitride layer can be removed with a wet etch solution such as a standard clean 1 (SC1) solution. In one or more embodiments, this stage can be used as the sensing structure 120.

In view 840, a sensing metal 812 is deposited and connected to the gate (e.g., conductive material 112) of the transducer 110. Example materials of the sensing metal 812 can include titanium oxide (TiOₓ), titanium nitride (TiN), platinum (Pt), gold (Au), etc.

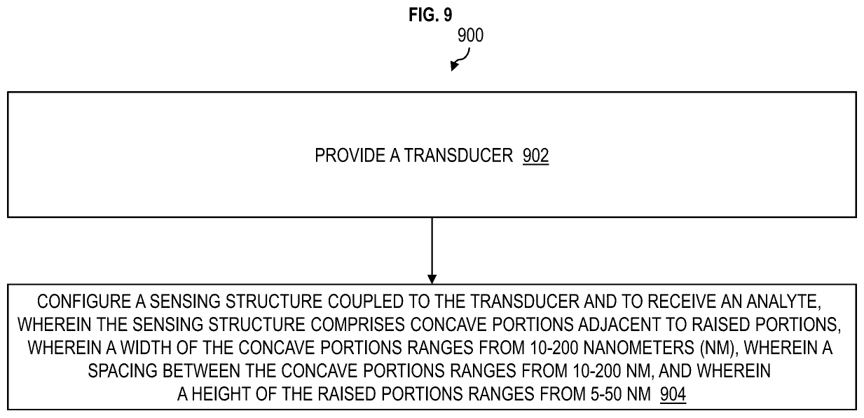

According to one or more embodiments, FIG. 9 depicts are method 900 of configuring a biosensor 100. Reference can be made to any figures discussed herein. At blocks 902 and 904, the method 900 includes providing a transducer 110 and configuring a sensing structure 120 to be coupled to the transducer 110 and to receive an analyte (e.g., a test solution). The sensing structure 120 comprises concave portions (e.g., cavities 210, 310, 410, 510, 610, 710, and 810) adjacent to raised portions (e.g., pillars 208 and 308, or fins 408, 508, 608, and 708), where a width of the concave portions ranges from 10-200 nanometers (nm), wherein a spacing between the concave portions ranges from 10-200 nm, and wherein a height of the raised portions ranges from 5-50 nm.

Also, the raised portions comprise a first material (e.g., second layer 204); a bottom of the concave portions comprises a second material (e.g., first layer 202) different from the first material; the width of the concave portions ranges from 10-100 nm; and the spacing between the concave portions ranges from 10-100 nm. An upper portion of the raised portions further comprises a third material (e.g., third layer 206), the third material being different from the first material. The second and third materials (e.g., first and third layers 202 and 206) are the same. The first material (e.g., second layer 204) comprises ruthenium oxide; and the second material (e.g., first layer 202) comprises at least one of platinum, gold, or titanium nitride.

The concave portions and the raised portions comprise a conducting material. The conducting material comprises titanium nitride, gold, or platinum. The concave portions are cylindrical cavities, for example, as depicted in FIGS. 2A, 2B, 3A, 3B, and 8.

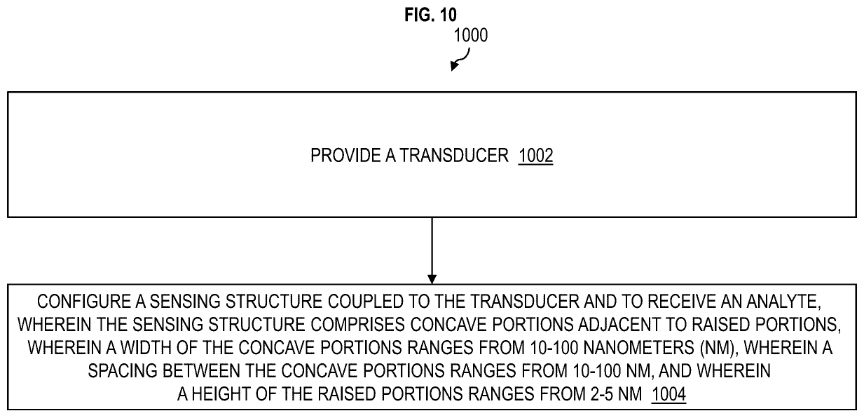

According to one or more embodiments, FIG. 10 depicts are method 1000 of configuring a biosensor 100. Reference can be made to any figures discussed herein. At blocks 1002 and 1004, the method 1000 includes providing a transducer 110 and configuring a sensing structure 120 to be coupled to the transducer and to receive an analyte (e.g., the test solution). The sensing structure 120 comprises concave portions (e.g., cavities 410, 510, 610, and 710) adjacent to raised portions (e.g., fins 408, 508, 608, and 708), where a width of the concave portions ranges from 10-100 nanometers (nm), wherein a spacing between the concave portions ranges from 10-100 nm, and wherein a height of the raised portions ranges from 2-5 nm.

Also, the concave portions are lateral channels, for example, as depicted in FIGS. 4A, 4B, 5A, 5B, 6A, 6B, 7A, and 7B.

Further, a curvature of the concave portions (e.g., cavities 410) comprises a first material (e.g., first layer 402); and the raised portions comprise the first material and a second material (e.g., second layer 404) on top of the first material, the second material being different from the first material. The first material (e.g., first layer 402) comprises a metal, and the second material (e.g., second layer 404) comprises a different metal or an insulator. The raised portions are fins 408. The first material (e.g., first layer 402) comprises titanium, titanium oxide, titanium nitride, ruthenium, ruthenium oxide, or cobalt.

According to one or more embodiments, the raised portions are fins 608 comprising a first material (e.g., second layer 604), the first material being formed on a second material (e.g., first layer 602); and the concave portions are lateral channels (e.g., fins 608) formed with a bottom of the second material (e.g., first layer 602). The raised portions further comprise a third material (e.g., third layer 606). The third material is a same as the second material (e.g., the first and the third layers 602 and 606 are the same material).

As discussed herein, gate material is formed around the semiconductor layers. The gate material includes high-k material and work function material generally referred to as a high-k metal gate (HKMG). Techniques for forming HKMG in gate openings are well-known in the art and, thus, the details have been omitted in order to allow the reader to focus on the salient aspects of the disclosed methods. However, it should be understood that such HKMG will generally include formation of one or more gate dielectric layers (e.g., an inter-layer (IL) oxide and a high-k gate dielectric layer), which are deposited so as to line the gate openings, and formation of one or more metal layers, which are deposited onto the gate dielectric layer(s) so as to fill the gate openings. The materials and thicknesses of the dielectric and metal layers used for the HKMG can be preselected to achieve desired work functions given the conductivity type of the FET. To avoid clutter in the drawings and to allow the reader to focus on the salient aspects of the disclosed methods, the different layers within the HKMG stack are not illustrated. For explanation purposes, a high-k gate dielectric layer can be, for example, a dielectric material with a dielectric constant that is greater than the dielectric constant of silicon dioxide (i.e., greater than 3.9). Exemplary high-k dielectric materials include, but are not limited to, hafnium (Hf)-based dielectrics (e.g., hafnium oxide, hafnium silicon oxide, hafnium silicon oxynitride, hafnium aluminum oxide, etc.) or other suitable high-k dielectrics (e.g., aluminum oxide, tantalum oxide, zirconium oxide, etc.). Optionally, the metal layer(s) can include a work function metal that is immediately adjacent to the gate dielectric layer and that is preselected in order to achieve an optimal gate conductor work function given the conductivity type of the nanosheet-FET. For example, the optimal gate conductor work function for the PFETs can be, for example, between about 4.9 eV and about 5.2 eV. Exemplary metals (and metal alloys) having a work function within or close to this range include, but are not limited to, ruthenium, palladium, platinum, cobalt, and nickel, as well as metal oxides (aluminum carbon oxide, aluminum titanium carbon oxide, etc.) and metal nitrides (e.g., titanium nitride, titanium silicon nitride, tantalum silicon nitride, titanium aluminum nitride, tantalum aluminum nitride, etc.). The optimal gate conductor work function for NFETs can be, for example, between 3.9 eV and about 4.2 eV. Exemplary metals (and metal alloys) having a work function within or close to this range include, but are not limited to, hafnium, zirconium, titanium, tantalum, aluminum, and alloys thereof, such as, hafnium carbide, zirconium carbide, titanium carbide, tantalum carbide, and aluminum carbide. The metal layer(s) can further include a fill metal or fill metal alloy, such as tungsten, a tungsten alloy (e.g., tungsten silicide or titanium tungsten), cobalt, aluminum, or any other suitable fill metal or fill metal.

Although not shown in detail, contact formation and ILD formation are performed. As such, ILD material can be deposited, source/drain contact openings are patterned by conventional lithography, and then metal is deposited to fill the cavities thereby forming metal contacts. A portion of the metal contacts may include silicide, resulting from the interface of the metal material and semiconductor material. The metal contacts are source/drain contacts that are respectively connected to epitaxial source/drain regions.

The ILD material can be SiO₂, SiN, a low-k dielectric material or an ultra-low-k dielectric material. Low-k dielectric materials may generally include dielectric materials having a k value of about 3.9 or less. The ultra-low-k dielectric material generally includes dielectric materials having a k value less than 2.5. Unless otherwise noted, all k values mentioned in the present application are measured relative to a vacuum. Exemplary ultra-low-k dielectric materials generally include porous materials such as porous organic silicate glasses, porous polyamide nanofoams, silica xerogels, porous hydrogen silsequioxane (HSQ), porous methylsilsesquioxane (MSQ), porous inorganic materials, porous CVD materials, porous organic materials, or combinations thereof. The ultra-low-k dielectric material can be produced using a templated process or a sol-gel process as is generally known in the art. In the templated process, the precursor typically contains a composite of thermally labile and stable materials. After film deposition, the thermally labile materials can be removed by thermal heating, leaving pores in the dielectric film. In the sol gel process, the porous low-k dielectric films can be formed by hydrolysis and polycondensation of an alkoxide(s) such as tetraetehoxysilane (TEOS).

Various embodiments of the present invention are described herein with reference to the related drawings. Alternative embodiments can be devised without departing from the scope of this invention. Although various connections and positional relationships (e.g., over, below, adjacent, etc.) are set forth between elements in the following description and in the drawings, persons skilled in the art will recognize that many of the positional relationships described herein are orientation-independent when the described functionality is maintained even though the orientation is changed. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. As an example of an indirect positional relationship, references in the present description to forming layer “A” over layer “B” include situations in which one or more intermediate layers (e.g., layer “C”) is between layer “A” and layer “B” as long as the relevant characteristics and functionalities of layer “A” and layer “B” are not substantially changed by the intermediate layer(s).

The phrase “selective to,” such as, for example, “a first element selective to a second element,” means that the first element can be etched and the second element can act as an etch stop.

As used herein, “p-type” refers to the addition of impurities to an intrinsic semiconductor that creates deficiencies of valence electrons. In a silicon-containing substrate, examples of p-type dopants, i.e., impurities, include but are not limited to: boron, aluminum, gallium and indium.

As used herein, “n-type” refers to the addition of impurities that contributes free electrons to an intrinsic semiconductor. In a silicon containing substrate examples of n-type dopants, i.e., impurities, include but are not limited to antimony, arsenic and phosphorous.

As previously noted herein, for the sake of brevity, conventional techniques related to semiconductor device and integrated circuit (IC) fabrication may or may not be described in detail herein. By way of background, however, a more general description of the semiconductor device fabrication processes that can be utilized in implementing one or more embodiments of the present invention will now be provided. Although specific fabrication operations used in implementing one or more embodiments of the present invention can be individually known, the described combination of operations and/or resulting structures of the present invention are unique. Thus, the unique combination of the operations described in connection with the fabrication of a semiconductor device according to the present invention utilize a variety of individually known physical and chemical processes performed on a semiconductor (e.g., silicon) substrate, some of which are described in the immediately following paragraphs.

In general, the various processes used to form a micro-chip that will be packaged into an IC fall into four general categories, namely, film deposition, removal/etching, semiconductor doping and patterning/lithography. Deposition is any process that grows, coats, or otherwise transfers a material onto the wafer. Available technologies include physical vapor deposition (PVD), chemical vapor deposition (CVD), electrochemical deposition (ECD), molecular beam epitaxy (MBE) and more recently, atomic layer deposition (ALD) among others. Removal/etching is any process that removes material from the wafer. Examples include etch processes (either wet or dry), and chemical-mechanical planarization (CMP), and the like. Semiconductor doping is the modification of electrical properties by doping, for example, transistor sources and drains, generally by diffusion and/or by ion implantation. These doping processes are followed by furnace annealing or by rapid thermal annealing (RTA). Annealing serves to activate the implanted dopants. Films of both conductors (e.g., poly-silicon, aluminum, copper, etc.) and insulators (e.g., various forms of silicon dioxide, silicon nitride, etc.) are used to connect and isolate transistors and their components. Selective doping of various regions of the semiconductor substrate allows the conductivity of the substrate to be changed with the application of voltage. By creating structures of these various components, millions of transistors can be built and wired together to form the complex circuitry of a modern microelectronic device.

As noted above, atomic layer etching processes can be used in the present invention for via residue removal, such as can be caused by via misalignment. The atomic layer etch process provide precise etching of metals using a plasma-based approach or an electrochemical approach. The atomic layer etching processes are generally defined by two well-defined, sequential, self-limiting reaction steps that can be independently controlled. The process generally includes passivation followed selective removal of the passivation layer and can be used to remove thin metal layers on the order of nanometers. An exemplary plasma-based approach generally includes a two-step process that generally includes exposing a metal such a copper to chlorine and hydrogen plasmas at low temperature (below 20 °C). This process generates a volatile etch product that minimizes surface contamination. In another example, cyclic exposure to an oxidant and hexafluoroacetylacetone (Hhfac) at an elevated temperature such as at 275 °C can be used to selectively etch a metal such as copper. An exemplary electrochemical approach also can include two steps. A first step includes surface-limited sulfidization of the metal such as copper to form a metal sulfide, e.g., Cu₂S, followed by selective wet etching of the metal sulfide, e.g., etching of Cu₂S in HCl. Atomic layer etching is relatively recent technology and optimization for a specific metal is well within the skill of those in the art. The reactions at the surface provide high selectivity and minimal or no attack of exposed dielectric surfaces.

Semiconductor lithography is the formation of three-dimensional relief images or patterns on the semiconductor substrate for subsequent transfer of the pattern to the substrate. In semiconductor lithography, the patterns are formed by a light sensitive polymer called a photoresist. To build the complex structures that make up a transistor and the many wires that connect the millions of transistors of a circuit, lithography and etch pattern transfer steps are repeated multiple times. Each pattern being printed on the wafer is aligned to the previously formed patterns and slowly the conductors, insulators and selectively doped regions are built up to form the final device.

The photoresist can be formed using conventional deposition techniques such chemical vapor deposition, plasma vapor deposition, sputtering, dip coating, spin-on coating, brushing, spraying and other like deposition techniques can be employed. Following formation of the photoresist, the photoresist is exposed to a desired pattern of radiation such as X-ray radiation, extreme ultraviolet (EUV) radiation, electron beam radiation or the like. Next, the exposed photoresist is developed utilizing a conventional resist development process.

After the development step, the etching step can be performed to transfer the pattern from the patterned photoresist into the interlayer dielectric. The etching step used in forming at least one opening can include a dry etching process (including, for example, reactive ion etching, ion beam etching, plasma etching or laser ablation), a wet chemical etching process or any combination thereof.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

In some embodiments, various functions or acts can take place at a given location and/or in connection with the operation of one or more apparatuses or systems. In some embodiments, a portion of a given function or act can be performed at a first device or location, and the remainder of the function or act can be performed at one or more additional devices or locations.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The present disclosure has been presented for the purposes of illustration and description but is not intended to be exhaustive or limited to the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

The diagrams depicted herein are illustrative. There can be many variations to the diagrams or the steps (or operations) described therein without departing from the spirit of the disclosure. For instance, the actions can be performed in a differing order or actions can be added, deleted or modified. Also, the term “coupled” describes having a signal path between two elements and does not imply a direct connection between the elements with no intervening elements/connections therebetween. All of these variations are considered a part of the present disclosure.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” are understood to include any integer number greater than or equal to one, i.e., one, two, three, four, etc. The terms “a plurality” are understood to include any integer number greater than or equal to two, i.e., two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A biosensor comprising:
a transducer; and
a sensing structure coupled to the transducer and configured to receive an analyte, wherein the sensing structure comprises concave portions adjacent to raised portions, wherein a width of the concave portions ranges from 10-200 nanometers (nm), wherein a spacing between the concave portions ranges from 10-200 nm, and wherein a height of the raised portions ranges from 5-50 nm.

2. The biosensor of claim 1, wherein:
the raised portions comprise a first material;
a bottom of the concave portions comprises a second material different from the first material;
the width of the concave portions ranges from 10-100 nm; and
the spacing between the concave portions ranges from 10-100 nm.

3. The biosensor of claim 2, wherein an upper portion of the raised portions further comprises a third material, the third material being different from the first material.

4. The biosensor of claim 3, wherein the second and third materials are a same.

5. The biosensor of claim 2, wherein:
the first material comprises ruthenium oxide; and
the second material comprises at least one of platinum, gold, or titanium nitride.

6. The biosensor of claim 1, wherein the concave portions and the raised portions comprise a conducting material.

7. The biosensor of claim 6, wherein the conducting material comprises titanium nitride, gold, or platinum.

8. The biosensor of claim 1, wherein the concave portions are cylindrical cavities.

9. A biosensor comprising:
a transducer; and
a sensing structure coupled to the transducer and configured to receive an analyte, wherein the sensing structure comprises concave portions adjacent to raised portions, wherein a width of the concave portions ranges from 10-200 nanometers (nm), wherein a spacing between the concave portions ranges from 10-100 nm, and wherein a height of the raised portions ranges from 2-5 nm.

10. The biosensor of claim 9, wherein the concave portions are lateral channels.

11. The biosensor of claim 9, wherein:
a curvature of the concave portions comprises a first material; and
the raised portions comprise the first material and a second material on top of the first material, the second material being different from the first material.

12. The biosensor of claim 11, wherein:
the first material comprises a metal; and
the second material comprises a different metal or an insulator.

13. The biosensor of claim 11, wherein the first material comprises titanium, titanium oxide, titanium nitride, ruthenium, ruthenium oxide, or cobalt.

14. The biosensor of claim 9, wherein:
the raised portions are fins comprising a first material, the first material being formed on a second material; and
the concave portions are lateral channels formed with a bottom of the second material.

15. The biosensor of claim 14, wherein the raised portions further comprise a third material.

16. The biosensor of claim 15, wherein the third material is a same as the second material.

17. A method for a biosensor comprising:
providing a transducer; and
providing a sensing structure coupled to the transducer and configured to receive an analyte, wherein the sensing structure comprises concave portions adjacent to raised portions, wherein a width of the concave portions ranges from 10-200 nanometers (nm), wherein a spacing between the concave portions ranges from 10-200 nm, and wherein a height of the raised portions ranges from 5-50 nm.

18. The method of claim 17, wherein:
the raised portions comprise a first material;
a bottom of the concave portions comprises a second material different from the first material;
the width of the concave portions ranges from 10-100 nm; and
the spacing between the concave portions ranges from 10-100 nm.

19. The method of claim 18, wherein an upper portion of the raised portions further comprises a third material, the third material being different from the first material.

20. The method of claim 19, wherein the second and third materials are a same.
